(19) United States
(12) Patent Application Publication
(10) Pub. No.: US 2019/0313981 A1
(43) Pub. Date: Oct. 17, 2019

(54) BLOOD PRESSURE MEASURING APPARATUS, SYSTEM, METHOD, AND PROGRAM

(71) Applicants: OMRON CORPORATION, Kyoto-shi (JP); OMRON HEALTHCARE CO., LTD., Kyoto (JP)

(72) Inventors: Natsuko HORIGUCHI, Kyoto-shi, Kyoto (JP); Hiroshi NAKAJIMA, Kyoto-shi, Kyoto (JP); Tomohiro KUKITA, Kyoto (JP); Hirotaka WADA, Kyoto-shi, Kyoto (JP); Tamio UEDA, Takatsuki-shi, Osaka (JP)

(21) Appl. No.: 16/454,362

(22) Filed: Jun. 27, 2019

Related U.S. Application Data
(63) Continuation of application No. PCT/JP2017/044396, filed on Dec. 11, 2017.

(30) Foreign Application Priority Data
Jan. 4, 2017 (JP) 2017-000240

(51) Int. Cl. A61B 5/00; A61B 5/0205; A61B 5/022

(52) U.S. Cl. CPC A61B 5/7289; A61B 5/0205; A61B 5/11; A61B 5/7221; A61B 5/02233

(57) ABSTRACT

A measuring apparatus includes a biological sensor that detects biological information, a movement sensor that detects movement information of a living body as a target of the biological information, a determination unit that determines whether the body starts to move and whether the body is moving, based on the biological information and the movement information, a recording unit that records the biological information until a common value is obtained, if it is determined that the body starts to move, an analyzer that determines whether a history of a biological value of the biological information from when the biological value starts to decrease until the biological value reaches the common value is within a proper range, and a deleting unit that deletes a corresponding biological information from the recording unit if the history is within the proper range.

### CROSS-REFERENCE TO RELATED APPLICATIONS

This application is a Continuation Application of PCT Application No. PCT/JP2017/044396, filed Dec. 11, 2017 and based upon and claiming the benefit of priority from Japanese Patent Application No. 2017-000240, filed Jan. 4, 2017, the entire contents of all of which are incorporated herein by reference.

### FIELD

The present invention relates to a blood pressure measuring apparatus, a system, a method, and a program for continuously measuring biological information.

### BACKGROUND

Regarding early detecting changes in a body utilizing biological information and using the information in medical treatment, an environment in which high performance can be easily used is made with developing a sensor technology, and thus importance of using the biological information in medical care also increases gradually.

For example, there is an apparatus that outputs an alarm in a case where it is detected that biological information has an abnormal value (for example, see Jpn. Pat. Appln. KOKAI Publication No. 2016-197777). A technology of estimating the need of a user with biological information is also proposed (for example, see Jpn. Pat. Appln. KOKAI Publication No. 2016-71716). A method of calculating the consumed calorie based on a pulse rate is also provided (for example, see Jpn. Pat. Appln. KOKAI Publication No. 9-294727).

In recent years, for example, a user terminal (for example, according to the tonometry method) capable of continuously measuring blood pressure of a user for each beat only by being worn on the wrist of the user has been realized. According to such a user terminal, it is possible to constantly measure the blood pressure without putting a heavy burden on the user.

### SUMMARY

According to a first aspect of the present invention, a blood pressure measuring apparatus includes a biological sensor that continuously detects biological information in time; a movement sensor that normally detects movement information of a living body as a target of the biological information; a determination unit that determines whether or not the living body starts to move and whether or not the living body is moving, by referring to the biological information and the movement information; a recording unit that records the biological information until a normal value is obtained, in a case where it is determined that the living body starts to move; an analyzer that determines whether or not a time history of a value of the biological information from when the value of the biological information starts to decrease until the value of the biological information reaches the normal value is within a proper range; and a deleting unit that deletes a corresponding biological information from the recording unit in a case where the time history is within the proper range.

According to a second aspect of the present invention, the analyzer presets a normal range of a time period from a time point at which the value of the biological information is a maximum value at time of motion until the value of the biological information comes back to the normal value in accordance with a difference between the maximum value and the normal value, determines that the time history is within the proper range in a case where the time period to come back from the maximum value to the normal value by the time history is within the normal range, and determines that the time history is not within the proper range in a case where the time period to come back from the maximum value to the normal value by the time history is not within the normal range.

According to a third aspect of the present invention, the analyzer presets a normal range of a magnitude of a gradient which refers an increment of a value per unit time in a time period to come back from a maximum value at time of motion to a value in a normal time by using a size of the maximum value and an elapsed time from a time point at which the maximum value has been recorded as variables, determines that the time history is within the proper range in a case where the magnitude of the gradient of each time in the time history is within the normal range, and determines that the time history is not within the proper range in a case where the magnitude of the gradient of each time in the time history is not within the normal range.

According to a fourth aspect of the present invention, the analyzer presets a normal range of a magnitude of a gradient which refers an increment of a value per unit time in a time period to come back from a maximum value at time of motion to a value in a normal time by using a size of the maximum value as a variable, in a time period from when the value of the biological information is the maximum value at time of motion until the value of the biological information comes back to a value in the normal time, determines that the time history is within the proper range in a case where magnitudes of all gradients in the time history are within the normal range, and determines that the time history is not within the proper range in a case where the magnitude of a gradient of a time in the time history is not within the normal range.

According to a fifth aspect of the present invention, the sensor detects blood pressure as the biological information.

According to the first aspect of the present invention, it is possible to normally acquire biological information of a living body and to acquire the biological information after motion. Accordingly, it is possible to suitably manage the state of a living body and to immediately warn a user in a case where an abnormal state has been detected. Further, in a case where the time history is within the proper range, the corresponding biological information is deleted from the recording unit. Thus, data obtained by recording the normal state becomes unnecessary in a state where abnormality is detected. Accordingly, it is possible to effectively utilize memory capacity of a memory and the like by deleting the unnecessary data. In a case where the time history is not within the proper range, since the recorded biological information is present in a state of being recorded, it is possible to specifically find a cause of abnormality occurring by examining the recorded biological information.

According to the second aspect of the present invention, it is possible to determine whether or not the time history is within the proper range, only by measuring the time to come back from the maximum value to the normal value. Accordingly, it is possible to easily perform determination, and the speed of a determination process increases. Accordingly, use efficiency of CPU resources used for the determination process increases.

According to the third aspect of the present invention, the normal range of the magnitude of the gradient for each time point is determined, and thus it is possible to obtain a determination result having high accuracy. Accordingly, it is possible to issue a more proper warning. Further, since it is possible to obtain more appropriate biological information, it is possible to more specifically find the cause of abnormality occurring.

According to the fourth aspect of the present invention, all the gradients until the value of the biological information comes back from the maximum value to the value in the normal time are collectively determined without considering each time point. Thus, it is possible to easily perform determination, and the speed of the determination process increases. Accordingly, use efficiency of CPU resources used for the determination process increases.

According to the fifth aspect of the present invention, it is possible to suitably manage the state of the living body and to immediately warn a user in a case where an abnormal state has been detected. Further, in a case where the time history is within the proper range, data of the corresponding blood pressure value in the time history is removed from the recording unit. Thus, data of a blood pressure value in the time history, in which a state of no abnormality has been recorded, becomes unnecessary in a state where abnormality is detected. Accordingly, it is possible to effectively utilize memory capacity of a memory and the like by deleting the unnecessary data. In a case where the time history is not within the proper range, since the data obtained by recording the blood pressure value in the time history is present in a state of being recorded, it is possible to more specifically find the cause of abnormality occurring by examining the data obtained by recording the blood pressure value in the time history.

That is, according to the aspects of the present invention, it is possible to provide a blood pressure measuring apparatus, a system, a method, and a program in which it is possible to continuously acquire biological information, to reliably determine and report an occurrence of abnormality in a living body, and to reduce the data volume of the biological information.

### BRIEF DESCRIPTION OF THE DRAWINGS

FIG. 1 is a block diagram illustrating a blood pressure measuring apparatus according to a first embodiment.

FIG. 2 is a diagram illustrating a watch-type wearable terminal as a specific example of the blood pressure measuring apparatus in FIG. 1.

FIG. 3 is a diagram illustrating that the blood pressure measuring apparatus in FIG. 1 is connected to a smart device, and the smart device is connected to a server.

FIG. 4 is a graph illustrating a time history curve of a blood pressure value of blood pressure as biological information, from when a motion starts until the blood pressure value comes back to a normal blood pressure value.

FIG. 5 is a flowchart illustrating an example of an operation of the blood pressure measuring apparatus in FIG. 1.

FIG. 6 is a block diagram illustrating a blood pressure measuring apparatus according to a second embodiment.

FIG. 7 is a block diagram illustrating a server according to the second embodiment.

### DETAILED DESCRIPTION

Hereinafter, a blood pressure measuring apparatus, a system, a method, and a program according to embodiments of the present invention will be described with reference to the drawings. In the following embodiments, units denoted by the same reference signs, and descriptions thereof will not be repeated.

The present embodiments have been made focusing on the above circumstances, and an object of the present embodiments is to provide a blood pressure measuring apparatus, a system, a method, and a program in which it is possible to continuously acquire biological information, to reliably determine and report a problem in a living body, and to reduce the data volume of biological information.

### First Embodiment

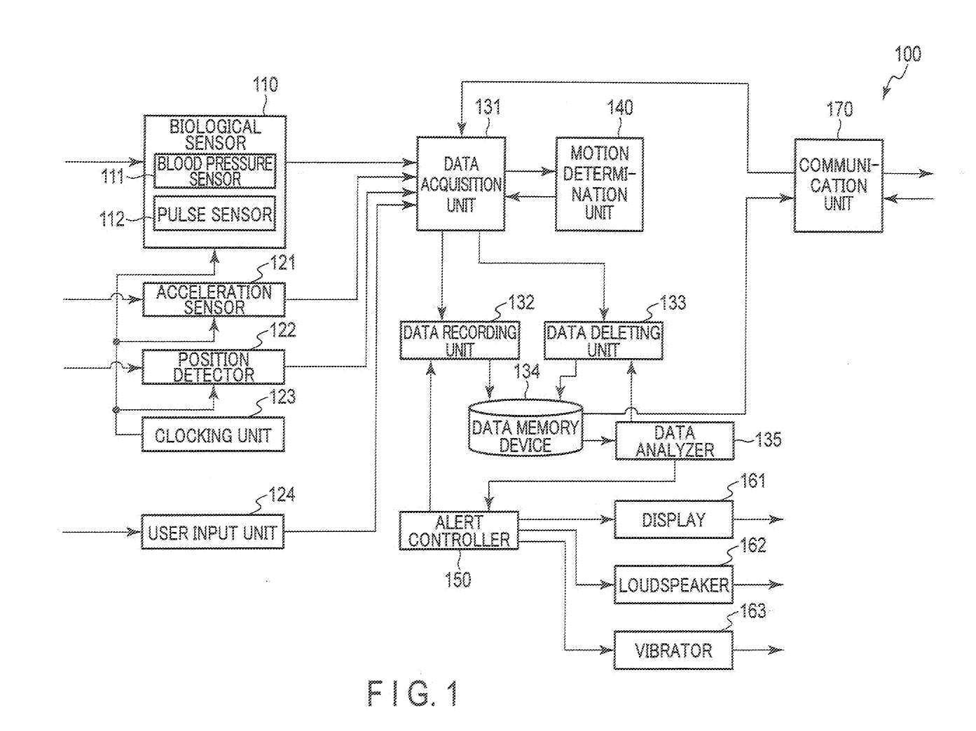

A blood pressure measuring apparatus 100 according to an embodiment will be described with reference to FIGS. 1 to 3.

The blood pressure measuring apparatus 100 includes a biological sensor 110, an acceleration sensor 121, a position detector 122, a clocking unit 123, a user input unit 124, a data acquisition unit 131, a data recording unit 132, a data deleting unit 133, a data memory device 134, a data analyzer 135, a motion determination unit 140, an alert controller 150, a display 161, a loudspeaker 162, a vibrator 163, and a communication unit 170. The biological sensor 110 includes a blood pressure sensor 111 and a pulse sensor 112.

The biological sensor 110 detects biological information from a living body, acquires a time point from the clocking unit 123, and outputs the biological information associated with the time point. Examples of the biological information include blood pressure and pulse. The blood pressure sensor 111 acquires blood pressure values from the living body and outputs the blood pressure values associated with time points which are continuously acquired from the clocking unit 123. The pulse sensor 112 acquires the pulses from the living body and outputs pulse values associated with the time points which are continuously acquired from the clocking unit 123. In the embodiment, the blood pressure sensor 111 and the pulse sensor 112 continuously detect biological information, and transfer detection data obtained by detection, for example, for 24 hours, to the data acquisition unit 131 at the next stage.

The acceleration sensor 121 is connected to the living body (for example, closely attached to the living body) to detect a movement of the living body. In the embodiment, the acceleration sensor 121 detects a three-axis acceleration of the living body and transfers the three-axis acceleration to the data acquisition unit 131 at the next stage.

The position detector 122 is connected to the living body (for example, closely attached to the living body) to detect a position of the living body. In the embodiment, the position detector 122 detects the position (latitude and longitude) of the living body with a global positioning system (GPS), WiFi, and/or Bluetooth (registered trademark) and transfers position information of the detected position to the data acquisition unit 131 at the next stage, along with the time point from the clocking unit 123.

The clocking unit 123 is configured to be capable of outputting the current time point and is, for example, a general clock. The clocking unit 123 may be set to be capable of acquiring time calibration information from the outside of the apparatus and outputting a correct time point.

The user input unit 124 acquires an instruction from a user and transfers an instruction signal of operating the blood pressure measuring apparatus 100 to the data acquisition unit 131. For example, the user input unit 124 receives ON and OFF of power from the user to turn the blood pressure measuring apparatus 100 ON and OFF.

The data acquisition unit 131 acquires data from the biological sensor 110, the acceleration sensor 121, the position detector 122, and the user input unit 124 and transfers a set of pieces of acquired data to the motion determination unit 140. The data acquisition unit transfers an instruction to the data recording unit 132 and/or the data deleting unit 133 based on a determination result of the motion determination unit 140.

The motion determination unit 140 determines whether or not the living body moves and then makes a motion, from the data from the data acquisition unit 131. The motion determination unit 140 continuously determines whether or not the living body moves and continues to move, for example, based on information from the acceleration sensor 121, in association with the time point. The motion determination unit 140 examines whether or not the pulse starts to increase, based on values of the pulse sensor 112 and determines that a possibility that the motion has been started is high if the pulse starts to increase. The motion determination unit 140 examines whether or not the position of the living body starts to move and then continues to move, from position data of the position detector 122. In a case where the moving is provided, the motion determination unit determines that the possibility that the motion has started is high. Further, in a case where the motion determination unit receives instruction data indicating that “a motion is started (motion has been started)” from the user input unit 124, the motion determination unit 140 determines that the possibility that the motion has been started is high. The motion determination unit 140 continuously determines whether or not the living body starts a motion and continues to move, based on at least above-described information, in association with the time point. For example, the motion determination unit 140 weights each type of data from the acceleration sensor 121, the pulse sensor 112, the position detector 122, and the user input unit 124, and determines that the living body is moving in a case where a value obtained by weighting the data is more than a predetermined value.

As another method, the motion determination unit 140 prioritizes each type of data from the acceleration sensor 121, the pulse sensor 112, the position detector 122, and the user input unit 124 and determines whether or not the living body is moving, based on one or more determination results of data having a high priority. For example, in a case where the motion determination unit 140 prioritizes the user input unit 124, the acceleration sensor 121, the pulse sensor 112, and the position detector 122 in this order, if an input from the user input unit 124 is received, the motion determination unit 140 determines whether or not the living body is moving, based on the above data. In this case, for example, in a case where the input from the user input unit 124 has not been received, the motion determination unit 140 performs the determination based on the acceleration sensor 121 having the next priority. In this case, in a case where data from the acceleration sensor 121 has not been provided, the motion determination unit 140 determines whether or not the living body is moving, based on the pulse sensor 112 having the next priority. Further, in a case where data of the pulse sensor 112 is not provided, the motion determination unit 140 determines whether or not the living body is moving, by the position detector 122 having the next priority.

The data recording unit 132 receives whether or not the living body starts to move, from the motion determination unit 140 via the data acquisition unit 131, for example. In a case where it is determined that the living body has moved, the data recording unit 132 starts to record data (biological information, for example, blood pressure) acquired from the biological sensor 110 by the data acquisition unit 131, in the data memory device 134. The data recording unit 132 records the biological information acquired from the biological sensor 110 by the data acquisition unit 131 in the data memory device 134 along with the time, in a time period in which the motion determination unit 140 has determined that the living body is moving. The data recording unit 132 may also record, for example, information from the acceleration sensor 121 and/or the position detector 122 in the data memory device 134 along with the time, in addition to the biological information from the biological sensor 110.

The data recording unit 132 records information in the data memory device 134, for example, until the blood pressure value comes back to a normal value, and stops recording of the information if the blood pressure value reaches the normal value.

The data recording unit 132 may compress data and store the compressed data, until the blood pressure value comes back to the normal value. In this case, the data volume becomes smaller by the compression, and thus a load on a communication is reduced. A generally known method may be used as a compression method. In addition, the data recording unit 132 may not record data as it is until the blood pressure value comes back to the normal value, but may record only features of the data so as to reduce the data volume while securing that the data can be reproduced later. For example, the data recording unit 132 records (1) the maximum value of the blood pressure value and a time point at this time, (2) the value at an inflection point of the time history curve of the blood pressure value and a time point at this time, and (3) the blood pressure value and a time point when the blood pressure value has come back to the normal value. When an attempt to reproduce the data is made, if it is predetermined that the above data is input in an order of (1), (2), and (3), along with a reproduction side, it is possible to reproduce the time history curve of the original blood pressure value on the reproduction side.

The data memory device 134 stores at least the biological information received from the biological sensor 110 along with the time in accordance with an instruction from the data recording unit 132. In a case where the data memory device has received an instruction to delete the designated data from the data deleting unit 133, the data memory device 134 deletes the designated data.

For example, in a case where the data deleting unit has received an instruction to delete the designated data from the user input unit 124, the data deleting unit 133 may delete the designated data from the data memory device 134.

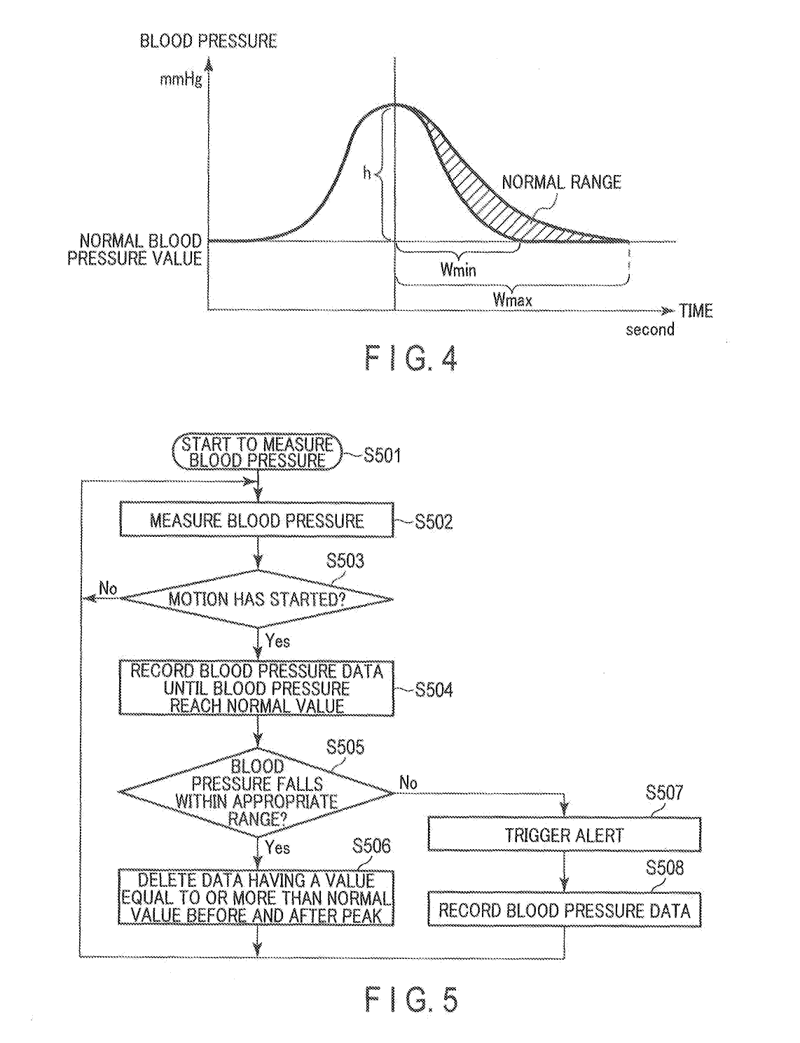

The data analyzer 135 acquires a time history of the biological information stored in the data memory device 134 and analyzes the biological information in a time period in which the living body is moving. The data analyzer 135 analyzes the degree of the biological information changing with the time. The data analyzer 135 determines whether or not a curve of the blood pressure value over time, for example, in a time period until the blood pressure in a motion comes back from the maximum value to the normal value is within aa proper range. In the embodiment, in a case where the curve of the blood pressure in the motion over time is within the proper range, for example, the data analyzer 135 instructs the data deleting unit 133 to delete the corresponding data of the blood pressure over time. In a case where the curve of the pressure over time, when the living body is moving, is not within the proper range, for example, the data analyzer transmits a message indicating that the blood pressure value is not normal, to the alert controller 150 without deleting the data. The analysis and the determination of the data analyzer 135 will be described later with reference to FIG. 4.

In a case where the data analyzer 135 determines that the curve of the blood pressure over time, when the living body is moving, is not within the proper range, the alert controller 150 transmits a signal of informing, for example, the user of a warning indicating that the time history of the blood pressure value is not normal, to at least one of the display 161, the loudspeaker 162, and the vibrator 163.

The display 161 receives the warning indicating that the time history of the blood pressure value is not normal, from the alert controller 150 and displays the warning. For example, the display 161 displays that “the blood pressure value is abnormal”.

The loudspeaker 162 receives the warning indicating that the time history of the blood pressure value is not normal, from the alert controller 150 and outputs the warning by voice. For example, the loudspeaker 162 outputs a voice of “the blood pressure value is abnormal” or outputs a warning sound (for example, buzzer sound).

The vibrator 163 receives the warning indicating that the time history of the blood pressure value is not normal, from the alert controller 150 and outputs the warning to the user by vibrating the blood pressure measuring apparatus 100 of the main body and adjuncts. The blood pressure measuring apparatus 100 is connected to the adjuncts vibrating with the blood pressure measuring apparatus 100 wirelessly or by wire. The user can recognize the warning in a manner that the adjunct receives the warning of the alert controller 150 from the blood pressure measuring apparatus 100, and the adjunct itself vibrates.

For example, the communication unit 170 transmits data stored in the data memory device 134 to an external server or receives an instruction to start or end the blood pressure measuring apparatus 100 from an external device.

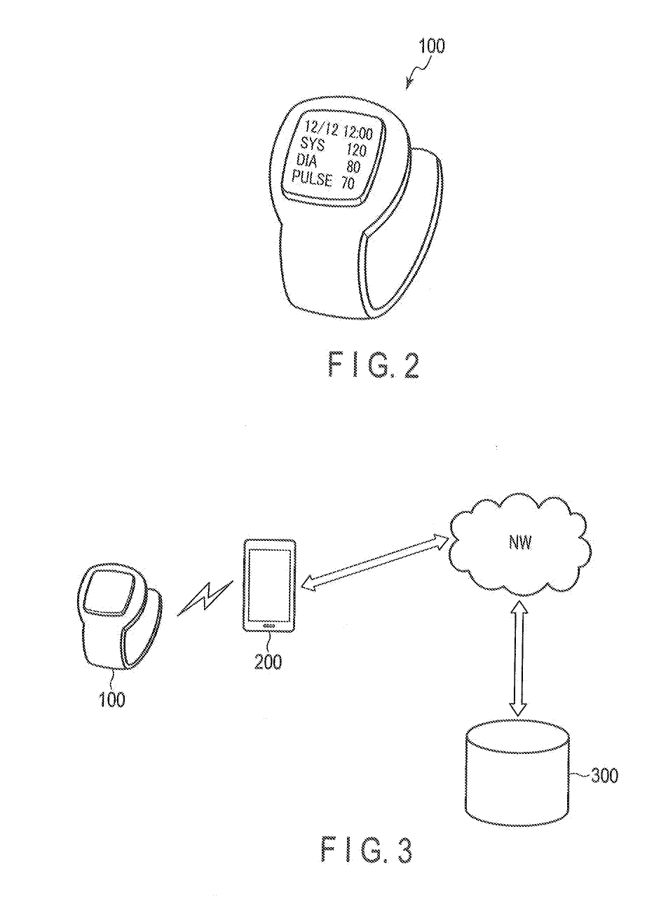

Next, a specific example of the blood pressure measuring apparatus 100 and cooperation with other devices will be described with reference to FIGS. 2 and 3.

The blood pressure measuring apparatus 100 may have any form and may be a watch-type wearable terminal illustrated in FIG. 2, for example. For example, the blood pressure measuring apparatus 100 displays biological information of the user, such as systolic blood pressure (SYS), diastolic blood pressure (DIA), and a pulse rate PULSE, in addition to the date of today and information such as the current time point, which is displayed in a general clock. The blood pressure measuring apparatus 100 can continuously measure the biological information of the user, for example, for each beat and display the latest SYS and DIA.

As illustrated in FIG. 3, the blood pressure measuring apparatus 100 may be connected to the smart device (typically, smartphone and tablet) 200. The smart device 200 displays a graph of state data transmitted by the blood pressure measuring apparatus 100 or transmits the state data to a server 300 via a network NW. Details of the state data will be described later. An application for managing the state data may be installed on the smart device 200.

The server 300 accumulates data transmitted from the blood pressure measuring apparatus 100 or the smart device 200. The server 300 may transmit data of the biological information of the user in response to an access from a personal computer (PC) and the like installed at a medical institution, for example, in order to provide the data of the biological information for health guidance or diagnosis for the user.

As will be described later, the server 300 may be a server 700 in a second embodiment. In this case, the server 300 may include the data analyzer 135 and the motion determination unit 140. The server 300 performs transmission to the blood pressure measuring apparatus 100 or the smart device 200 in order to perform display to the user.

Alternatively, the smart device 200 may include the data analyzer 135 and the motion determination unit 140. In this case, the smart device 200 transmits data to be displayed in the blood pressure measuring apparatus 100 in order to perform display to the user. The smart device 200 may display data.

Next, an operation in which the data analyzer 135 analyzes the degree of the biological information (here, blood pressure) changing with time and determines whether or not the curve (also referred to as the time history curve) of the blood pressure value over time in a time period until the blood pressure in the motion comes back from the maximum value to the normal value is within the proper range will be described with reference to FIG. 4.

In the embodiment, in a case where the blood pressure is handled as the biological information, the data analyzer 135 analyzes and determines whether or not the curve of the blood pressure value over time in the time period until the blood pressure in the motion comes back from the maximum value to the normal value is within the proper range. Some methods of analyzing whether or not the curve is within the proper range are assumed. Here, three methods will be described.

First Method: a normal range in a time period from a time point of the maximum blood pressure value until the blood pressure comes back to a normal blood pressure value can be predetermined in accordance with a difference (h in FIG. 4) between the maximum blood pressure value at time of motion and a normal blood pressure value. Thus, if the data analyzer 135 holds a table, a function, or the like (here, collectively referred to as a table) indicating a relation between the difference (h in FIG. 4) between the maximum blood pressure value at time of motion and the normal blood pressure value and a time width (Wmin and Wmax) being the proper range from the time point of the maximum blood pressure value until the blood pressure comes back to the normal blood pressure value, in advance, the data analyzer 135 can determine whether or not the measured data of the blood pressure in the time history is within the proper range.

Specifically, the data analyzer 135 acquires data of the blood pressure in the time history from the data memory device 134 and calculates the difference (h in FIG. 4) between the maximum blood pressure value at time of motion and the normal blood pressure value. The data analyzer obtains Wmin and Wmax when the calculated value (h in FIG. 4) has been obtained, from a table made based on the calculated value by referring to the table. The data analyzer 135 obtains a time width from the time point of the maximum blood pressure value at time of motion to a time point at which the blood pressure value has come back to the normal blood pressure value, from the data of the blood pressure in the time history in the data memory device 134. Then, the data analyzer determines whether or not the obtained time width is in a range between Wmin and Wmax obtained from the table. In a case where the time width is in the range between Wmin and Wmax obtained from the table, the data analyzer determines that the time history of the blood pressure is within the proper range, and thus is not abnormal. In a case where the time width is not in the above range, the data analyzer determines that the time history of the blood pressure is not normal.

According to the first method, it is possible to determine whether or not to be within the proper range only by measuring a time period to come back from the maximum value to the normal value. Thus, it is possible to easily perform the determination, and the speed of a determination process increases. Accordingly, use efficiency of CPU resources used for the determination process increases.

Second Method: a magnitude of a gradient which refers an increment of the blood pressure value per unit time in a time period to come back from the maximum blood pressure value at time of motion to the normal blood pressure value depends on the size of the maximum blood pressure value and an elapsed time from a time point at which the maximum blood pressure value has been recorded. Thus, if the data analyzer 135 holds a table which can be obtained by using the size of the maximum blood pressure value and the elapsed time at a time point at which the maximum blood pressure value has been recorded, as variables, in advance, the data analyzer 135 can determine whether or not the measured data of the blood pressure in the time history is within the proper range by obtaining the gradient from the data of the blood pressure in the time history. In this case, the gradient normally has a negative value since a case where the blood pressure value decreases is assumed. Thus, only the magnitude of the gradient is handled here, but the magnitude of the gradient with a sign is essentially the same as the above magnitude of the gradient even though the magnitude of the gradient with the sign is handled.

Specifically, the data analyzer 135 acquires the data of the blood pressure in the time history from the data memory device 134 and calculates the difference (h in FIG. 4) between the maximum blood pressure value at time of motion and the normal blood pressure value. The data analyzer determines whether or not the gradient at any time point in the time period from the time point at which the maximum blood pressure value has been recorded until the blood pressure value comes back to the normal blood pressure value is within the proper range, at some time points. Ideally, the data analyzer determines whether or not the gradients of all time points included in the data of the blood pressure in the time history from the data memory device 134 are within the proper range. However, the effect is obtained even if the determination is performed only at the first time point (time point at which the blood pressure value has been the maximum blood pressure value at time of motion), the last time point (time point at which the blood pressure value has come back to the normal blood pressure value), and some points of a plurality of intermediate points. In a case where the magnitudes of all the gradients at all the time points at which the data analyzer has determined whether or not the magnitude of the gradient is within the proper range are within the proper range, it is determined that the data of the blood pressure in the time history is within the proper range, and thus is not abnormal. In a case where the magnitudes of all the gradients are not within the proper range, it is determined that the time history of the blood pressure is not normal.

According to the second method, the normal range of the magnitude of the gradient for each time point is determined, and thus it is possible to obtain a determination result having high accuracy. Accordingly, it is possible to issue a more proper warning. Further, since it is possible to obtain more appropriate biological information, it is possible to more specifically find the cause of abnormality occurring.

Third Method: a third method is simpler than the second method. The magnitude of the gradient being the increment of the blood pressure value per unit time in a time period to come back from the maximum blood pressure value at time of motion to the normal blood pressure value depends on the size of the maximum blood pressure value and an elapsed time from a time point at which the maximum blood pressure value has been recorded. In the third method, only a point that the magnitude of the gradient in the proper range from a time point at which the maximum blood pressure value at time of motion has been measured until the blood pressure value comes back to the normal blood pressure value depends on the maximum blood pressure value at time of motion is used. Thus, the data analyzer 135 may hold the table of a lower limit value and an upper limit value of the magnitude of the gradient in the proper range from the time point at which the maximum blood pressure value at time of motion has been measured until the blood pressure value comes back to the normal blood pressure value, in advance, the magnitude of the gradient depending on the maximum blood pressure value at time of motion. The data analyzer 135 may calculate the magnitudes of all the gradients in the time period from the time point at which the maximum blood pressure value at time of motion has been measured until the blood pressure value comes back to the normal blood pressure value, from the data of the blood pressure acquired from the data memory device 134 and may determine whether the magnitudes of all the gradients are values between the lower limit value and the upper limit value of the magnitude of the gradient in the proper range.

According to the third method, all the gradients until the value of the biological information comes back from the maximum value to the value in the normal time are collectively determined without considering each time point. Thus, it is possible to easily perform determination, and the speed of the determination process increases. Accordingly, use efficiency of CPU resources used for the determination process increases.

Specifically, the data analyzer 135 acquires the data of the blood pressure in the time history from the data memory device 134 and calculates the difference (h in FIG. 4) between the maximum blood pressure value at time of motion and the normal blood pressure value. The data analyzer holds a table between the lower limit value and the upper limit value of the magnitude of the gradient in the proper range from the time point at which the maximum blood pressure value has been recorded until the blood pressure value comes back to the normal blood pressure value, in advance. The data analyzer determines whether or not the magnitude of the gradient in the data from the data memory device 134 is in the range between the lower limit value and the upper limit value. In a case where the magnitudes of all the gradients are in the range between the lower limit value and the upper limit value, the data analyzer determines that the data of the blood pressure in the time history is within the proper range, and thus is not abnormal. In a case where the magnitudes of all the gradients are not in the above range, the data analyzer determines that the time history of the blood pressure is not normal.

Here, although there are some methods of obtaining the magnitude of the gradient from the data of the data memory device 134, any method is used. As a simple method, regarding the gradient of the blood pressure from the data, a change rate of the blood pressure value between the blood pressure value at a certain time point and the blood pressure value at a time point in the vicinity of the above time point by using the time as a variable may be set as the gradient at this time point. In addition, a method of obtaining the gradient in a manner that a time-differentiable function (blood pressure value over time) that matches with the distribution of the blood pressure value with respect to the time point as much as possible is calculated, and this function is differentiated is also considered.

Next, the operation of the blood pressure measuring apparatus 100 will be described with reference to FIG. 5.

(Step S501) The blood pressure measuring apparatus 100 starts to measure blood pressure of a living body as a target. That is, the blood pressure sensor 111 starts to measure the blood pressure of the living body. If the data acquisition unit 131 detects that the pulse sensor 112 starts to acquire the pulse, the measurement starts. If a user causes the power of the blood pressure measuring apparatus 100 to turn ON by the user input unit 124, the measurement of the blood pressure may be started. In the embodiment, the blood pressure measuring apparatus 100 requires measurement of the blood pressure at time of motion. Thus, the measurement may be started if the acceleration sensor 121 and/or the position detector 122 have detected that the living body has moved. For example, in a case where the acceleration sensor 121 detects a three-axis acceleration, it is determined that the living body has moved in a case where an acceleration of any axis has become more than a preset threshold value. In addition, in a case where moving in latitude and longitude has become larger than a preset threshold value by the position detector 122, it is determined that the living body has moved. Determination criteria for determining that the living body has moved may be provided with a combination of the characteristics of the living body or all conditions by the sensors included in the blood pressure measuring apparatus 100.

(Step S502) The blood pressure sensor 111 of the biological sensor 110 continuously measures the blood pressure. In the embodiment, in the blood pressure measuring apparatus 100, the blood pressure sensor 111 is a sensor capable of continuously measuring the blood pressure. The blood pressure sensor 111 continuously measures the blood pressure of the user for each beat only by wearing the blood pressure sensor on the wrist of the user and is capable of continuously measuring the blood pressure for 24 hours. In this step, the blood pressure sensor 111 measures the blood pressure and transfers data to the data acquisition unit 131, and the motion determination unit 140 also receives the data. However, at this time point, it is not detected that the living body is moving. Thus, the value is not recorded in the data memory device 134. That is, the data of the blood pressure of the living body in the time history only after the living body moves is not recorded in the data memory device 134. Therefore, it is possible to record only necessary data of the blood pressure in the time history. An unnecessary burden on the capacity of the data memory device 134 is not provided. Thus, it is possible to efficiently utilize data resources.

(Step S503) The blood pressure measuring apparatus 100 determines whether or not the living body as a measurement target starts a motion. In a case where it is determined that the motion has been started, the process proceeds to Step S504. In a case where it is determined that the motion does not have been started, the process returns to Step S502, and the measurement continues. For example, in a case where the acceleration sensor 121 detects a three-axis acceleration, it is determined that the living body has moved in a case where an acceleration of any axis has become more than a preset threshold value. In addition, it may be determined whether or not the living body starts a motion by the position detector 122 and/or the pulse sensor 112 determining that the living body has moved. In this case, the determination can be performed by a method similar to that in Step S501.

(Step S504) The data memory device 134 stores the data (also referred to as blood pressure data) of the blood pressure in the time history in a time period in which the motion starts in Step S503 after blood pressure measurement in Step S501 starts, then the blood pressure increases and reaches the maximum value, and then the blood pressure reaches the normal value. Regarding the recognition that the blood pressure has reached the normal value, for example, the blood pressure is set to reach the normal value in a case where at least one or more recognitions of that it is recognized that an acceleration of any axis is less than a preset threshold value and that the position detector 122 recognizes that moving in latitude and longitude is smaller than a preset threshold value, in a case where the acceleration sensor 121 detects a three-axis acceleration in a state where the blood pressure sensor 111 has recognized that the blood pressure has reached the normal value. Since the blood pressure value at time at which the measurement starts in Step S501 is the normal value, the data memory device 134 stores the normal value via the data acquisition unit 131 and the data recording unit 132.

(Step S505) The data analyzer 135 determines whether or not a falling curve in which the blood pressure data which is measured and then is stored in the data memory device 134 increases by a motion and then falls from the maximum blood pressure value is within the proper range. Details are specifically made with reference to FIG. 4. In a case where it is determined that falling of the blood pressure is within the proper range, the process proceeds to Step S506. In a case where the falling of the blood pressure is not within the proper range, the process proceeds to Step S507.

(Step S506) In the embodiment, the descriptions are made focusing on a case where the blood pressure data is abnormal. Thus, in a case where the falling of the blood pressure is within the proper range, the blood pressure data is set not to attract attentions and is set not to be useful data. As a result, in a case where the data deleting unit 133 determines that the blood pressure data is not abnormal, the data analyzer 135 outputs an instruction to the data deleting unit 133 so as to delete the blood pressure data stored in the data memory device 134. More specifically, the blood pressure data in a time period in which the blood pressure data in which it has been determined that the falling of the blood pressure has been within the proper range increases from the normal blood pressure value by starting a motion and then comes back to the normal blood pressure value again through the maximum blood pressure value is a deletion target. In the example in FIG. 4, all pieces of data corresponding to a curve distributed over the normal blood pressure value are deletion targets.

(Step S507) As described with reference to FIG. 4, in a case where the data analyzer 135 determines that the falling of the blood pressure is not within the proper range, the data analyzer 135 instructs the alert controller 150 to trigger an alert that the blood pressure data is abnormal. The alert controller 150 instructs the display 161, the loudspeaker 162, and the vibrator 163 to trigger an alert. The alert controller 150 may select any of the display 161, the loudspeaker 162, and the vibrator 163 and perform an output. For example, in a case where a silent mode or the like is set by the user input unit 124, the alert controller 150 may cause the display 161 not the loudspeaker 162 to perform a display or may operate only the vibrator 163.

(Step S508) In the embodiment, the descriptions are made focusing on a case where the blood pressure data is abnormal. Thus, in a case where the falling of the blood pressure is not within the proper range, the blood pressure data is considered to attract attentions and is set to be useful data. Therefore, for example, the alert controller 150 instructs the data recording unit 132 to record the blood pressure data in the data memory device 134. The data has been already recorded in Step S504. However, in this step, the attribute is changed to make the record permanent. Alternatively, in Step S504, the data may be recorded in a storage device that temporarily stores data (for example, an access speed is high, but capacity is small). In Step S508, for example, the blood pressure data may be stored in a storage device having a slow access speed, higher reliability, and large capacity (the data memory device 134 may include the two types of storage devices). Step S508 may be deleted, and only the data memory device 134 in Step S504 may be provided without providing a plurality of storage devices.

According to the above first embodiment, it is possible to normally acquire biological information (data of the blood pressure in the time history) of the living body and to acquire biological information after a motion. Thus, it is possible to suitably manage the state of the living body and to immediately warn a user in a case where an abnormal state has been detected. Further, in a case where the time history is within the proper range, the corresponding biological information is deleted from the recording unit. Thus, data obtained by recording the normal state becomes unnecessary in a state where abnormality is detected. Accordingly, it is possible to effectively utilize memory capacity of a memory and the like by deleting the unnecessary data. In a case where the time history is not within the proper range, since the recorded biological information is present in a state of being recorded, it is possible to specifically find a cause of abnormality occurring by examining the recorded biological information.

### Second Embodiment

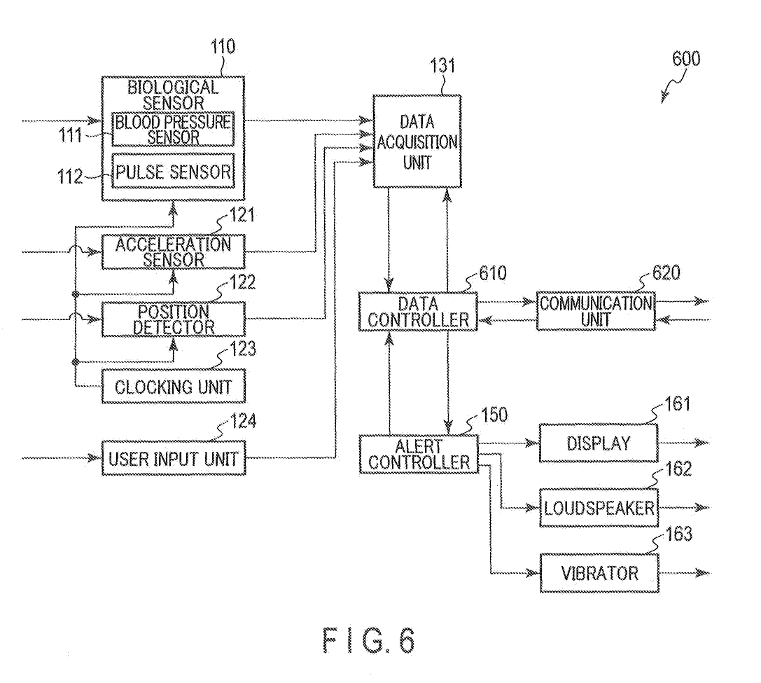

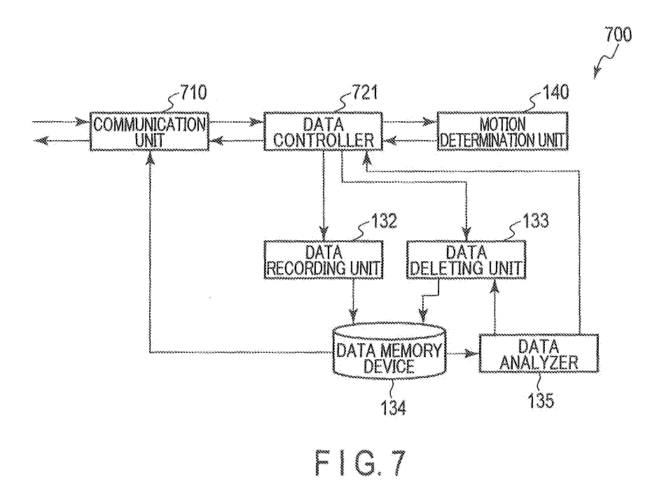

This embodiment is configured by a blood pressure measuring apparatus 600 in FIG. 6 and a server 700 in FIG. 7 and is obtained by modifying the blood pressure measuring apparatus 100 in the first embodiment. The blood pressure measuring apparatus is different from the blood pressure measuring apparatus 100 in the first embodiment that the blood pressure measuring apparatus 600 has only the minimum configuration, and the server 700 has other configurations.

In the embodiment, as illustrated in FIG. 6, the blood pressure measuring apparatus 600 includes a biological sensor 110, an acceleration sensor 121, a position detector 122, a clocking unit 123, a user input unit 124, a data acquisition unit 131, an alert controller 150, a display 161, a loudspeaker 162, a vibrator 163, a data controller 610, and a communication unit 620. The data controller 610 and the communication unit 620 are unique to the second embodiment.

In the embodiment, as illustrated in FIG. 7, the server 700 includes a communication unit 710, a data controller 721, a data recording unit 132, a data deleting unit 133, a data memory device 134, a data analyzer 135, and a motion determination unit 140. The communication unit 710 and the data controller 721 are unique to the second embodiment.

The data controller 610 transmits data (acquired by the data acquisition unit 131) from the biological sensor 110, the acceleration sensor 121, the position detector 122, and the user input unit 124 to the server 700 via the communication unit 620.

The communication unit 710 of the server 700 receives data from the data acquisition unit 131, and the data controller 721 transfers the data to the motion determination unit 140. The motion determination unit 140 determines whether or not a living body as a target of the blood pressure measuring apparatus 600 moves and then makes a motion. The data recording unit 132 receives whether or not the living body starts to move, from the motion determination unit 140 via the data acquisition unit 131, for example. In a case where it is determined that the living body has moved, the data recording unit 132 starts to record data (biological information, for example, blood pressure) acquired from the biological sensor 110 by the data acquisition unit 131, in the data memory device 134. Then, in a case where the curve of the pressure over time, when the living body is moving, is not within the proper range, for example, the data analyzer 135 does not delete the data, but transmits a message indicating that the blood pressure value is not normal, to the blood pressure measuring apparatus 600 from the communication unit 710 via the data controller 721.

The communication unit 620 of the blood pressure measuring apparatus 600 receives the message indicating that the blood pressure value is not normal. The alert controller 150 receives the message indicating that the blood pressure value is not normal via the data controller 610. The alert controller 150 transmits a signal of informing, for example, a user of a warning indicating that a time history of the blood pressure value is not normal to at least one of the display 161, the loudspeaker 162, and the vibrator 163.

The server 700 is the smart device 200 or the server 300 illustrated in FIG. 2, for example. The server 700 has a configuration illustrated in FIG. 7 and may be separated from the blood pressure measuring apparatus 600.

According to the above second embodiment, the blood pressure measuring apparatus 600 can be made with the minimum configuration. Thus, the apparatus worn by the user can be small and lightweight, and a design according to the preference of the user is easily made. Since the device portion of the blood pressure measuring apparatus 600 is reduced, the blood pressure measuring apparatus 600 can be provided at a lower price. Further, since the amount of calculation by the blood pressure measuring apparatus 600 is small, it is possible to reduce the memory amount and to reduce the CPU usage.

The apparatus in the present embodiments can also be realized by a computer and a program. The program can be recorded in a recording medium or can be provided through a network.

Each of the above apparatuses and the apparatus portions can be implemented by any of a hardware configuration and a combined configuration of hardware resources and software. As the software in the combined configuration, a program which is installed on a computer from a computer readable recording medium or a network in advance and is executed by a processor of the computer, and thus causes the computer to realize functions of the apparatus is used.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Some or all of the above embodiments are described as in the following notes but are not limited to the following notes.

### (Supplemental Note 1)

A blood pressure measuring apparatus comprising a hardware processor and a memory, wherein
the hardware processor is configured to
continuously detect biological information in time,
normally detect movement information of a living body as a target of the biological information,
determine whether or not the living body starts to move and whether or not the living body is moving, by referring to the biological information and the movement information,
record the biological information until a normal value is obtained, in a case where it is determined that the living body starts to move,
determine whether or not a time history of a value of the biological information from when the value of the biological information starts to decrease until the value of the biological information reaches the normal value is within a proper range, and
delete a corresponding biological information in a case where the time history is within the proper range, and the memory includes a storage unit that stores the biological information.

### (Supplemental Note 2)

A system comprising a blood pressure measuring apparatus and a server, the apparatus comprising a first hardware processor and a first memory, a server comprising a second hardware processor and a second memory, wherein
in the blood pressure measuring apparatus,
the first hardware processor is configured to continuously detect biological information in time and normally detect movement information of a living body as a target of the biological information,
the first memory is configured to store the biological information, and
in the server,
the second hardware processor is configured to determine whether or not the living body starts to move and whether or not the living body is moving, by referring to the biological information and the movement information,
the second memory is configured to record the biological information until a normal value is obtained, in a case where it is determined that the living body starts to move,
the second hardware processor is configured to determine whether or not a time history of a value of the biological information from when the value of the biological information starts to decrease until the value of the biological information reaches the normal value is within a proper range, and
in the blood pressure measuring apparatus, the first hardware processor is configured to delete the corresponding biological information in a case where the time history is within the proper range.

### (Supplemental Note 3)

A blood pressure measuring method comprising:
continuously detecting biological information in time by using at least one hardware processor,
normally detecting movement information of a living body as a target of the biological information by using at least one hardware processor,
determining whether or not the living body starts to move and whether or not the living body is moving, by referring to the biological information and the movement information by using at least one hardware processor, and
recording the biological information until a normal value is obtained, in a case where it is determined that the living body starts to move by using at least one hardware processor,
determining whether or not a time history of a value of the biological information from when the value of the biological information starts to decrease until the value of the biological information reaches the normal value is within a proper range by using at least one hardware processor, and
deleting the corresponding biological information in a case where the time history is within the proper range, by using at least one hardware processor.

What is claimed is:

1. A blood pressure measuring apparatus comprising:
a biological sensor configured to continuously detect biological information in time;
a movement sensor configured to normally detect movement information of a living body as a target of the biological information;
a determination unit configured to determine whether or not the living body starts to move and whether or not the living body is moving, by referring to the biological information and the movement information;
a recording unit configured to record the biological information until a normal value is obtained, in a case where it is determined that the living body starts to move;
an analyzer configured to determine whether or not a time history of a value of the biological information from when the value of the biological information starts to decrease until the value of the biological information reaches the normal value is within a proper range; and
a deleting unit configured to delete a corresponding biological information from the recording unit in a case where the time history is within the proper range.

2. The blood pressure measuring apparatus according to claim 1, wherein the analyzer presets a normal range of a time period from a time point at which the value of the biological information is a maximum value at time of motion until the value of the biological information comes back to the normal value in accordance with a difference between the maximum value and the normal value, determines that the time history is within the proper range in a case where the time period to come back from the maximum value to the normal value by the time history is within the normal range, and determines that the time history is not within the proper range in a case where the time period to come back from the maximum value to the normal value by the time history is not within the normal range.

3. The blood pressure measuring apparatus according to claim 1, wherein the analyzer presets a normal range of a magnitude of a gradient which refers an increment of a value per unit time in a time period to come back from a maximum value at time of motion to a value in a normal time by using a size of the maximum value and an elapsed time from a time point at which the maximum value has been recorded as variables, determines that the time history is within the proper range in a case where the magnitude of the gradient of each time in the time history is within the normal range, and determines that the time history is not within the proper range in a case where the magnitude of the gradient of each time in the time history is not within the normal range.

4. The blood pressure measuring apparatus according to claim 1, wherein the analyzer presets a normal range of a magnitude of a gradient which refers an increment of a value per unit time in a time period to come back from a maximum value at time of motion to a value in a normal time by using a size of the maximum value as a variable, in a time period from when the value of the biological information is the maximum value at time of motion until the value of the biological information comes back to a value in the normal time, determines that the time history is within the proper range in a case where magnitudes of all gradients in the time history are within the normal range, and determines that the time history is not within the proper range in a case where the magnitude of a gradient of a time in the time history is not within the normal range.

5. The blood pressure measuring apparatus according to claim 1, wherein the sensor detects blood pressure as the biological information.

6. The blood pressure measuring apparatus according to claim 1, further comprising an alert unit configured to output a warning indicating that the time history of the biological information is not normal, in a case where the time history is not within the proper range.

7. The blood pressure measuring apparatus according to claim 2, wherein the recording unit records the maximum value of the biological information in a time history curve and a time point at which the value of the biological information is the maximum value in the time history curve, a value of the biological information at an inflection point in the time history curve and a time point at which the value of the biological information is at the inflection point in the time history curve, and a value and a time point when the value of the biological information has come back to the normal value.

8. A blood pressure measuring method comprising:
continuously detecting biological information in time;
normally detecting movement information of a living body as a target of the biological information;
determining whether or not the living body starts to move and whether or not the living body is moving, by referring to the biological information and the movement information;
recording the biological information until a normal value is obtained, in a case where it is determined that the living body starts to move;
determining whether or not a time history of a value of the biological information from when the value of the biological information starts to decrease until the value of the biological information reaches the normal value is within a proper range; and
deleting the corresponding biological information in a case where the time history is within the proper range.

9. A system comprising: a blood pressure measuring apparatus that acquires biological information and outputs an alarm; and a server that performs analysis based on the biological information from the blood pressure measuring apparatus,
the blood pressure measuring apparatus comprising:
a biological sensor configured to continuously detect biological information in time; and
a movement sensor configured to normally detect movement information of a living body as a target of the biological information,
the server comprising:
a determination unit configured to determine whether or not the living body starts to move and whether or not the living body is moving, by referring to the biological information and the movement information;
a recording unit configured to record the biological information until a normal value is obtained, in a case where it is determined that the living body starts to move; and
an analyzer configured to determine whether or not a time history of a value of the biological information from when the value of the biological information starts to decrease until the value of the biological information reaches the normal value is within a proper range, and
the blood pressure measuring apparatus further comprising:
a deleting unit configured to delete a corresponding biological information from the recording unit in a case where the time history is within the proper range.

10. A non-transitory computer readable medium storing a computer program which is executed by a computer to provide the steps of:
continuously detecting biological information in time;
normally detecting movement information of a living body as a target of the biological information;
determining whether or not the living body starts to move and whether or not the living body is moving, by referring to the biological information and the movement information;
recording the biological information until a normal value is obtained, in a case where it is determined that the living body starts to move;
determining whether or not a time history of a value of the biological information from when the value of the biological information starts to decrease until the value of the biological information reaches the normal value is within a proper range; and
deleting the corresponding biological information in a case where the time history is within the proper range.
